(19) United States
(12) Patent Application Publication
(10) Pub. No.: US 2009/0125052 A1
(43) Pub. Date: May 14, 2009

(54) NASAL DILATOR

(75) Inventors: Fausto PINNA, Lesmo (IT); Marco Pinna, Arcisate (IT)

Correspondence Address: OBLON, SPIVAK, MCCLELLAND MAIER & NEUSTADT, P.C., 1940 DUKE STREET, ALEXANDRIA, VA, 22314, US

(73) Assignee: BIOFARMITALIA S.P.A., Milano (IT)

(21) Appl. No.: 12/256,777

(22) Filed: Oct. 23, 2008

(30) Foreign Application Priority Data
Nov. 13, 2007 (IT) MI2007A 002156

(51) Int. Cl. A61M 29/02; A61M 15/08

(52) U.S. Cl. 606/199; 128/200.24

(57) ABSTRACT

A nasal dilator consisting of an elongated elastically flexible element of plastic material having a layer of dermocompatible adhesive material applied onto a first surface thereof at each of its ends.

### FIELD OF THE INVENTION

The present invention relates to a nasal dilator, in particular a nasal dilator of plastic material having a surface partially covered by a dermocompatible adhesive layer to enable it to be fixed to the nose of a user, to widen the nostrils and facilitate breathing.

### BACKGROUND OF THE INVENTION

Adhesive strips able to dilate the user's nostrils are known, to facilitate respiration; they consist of numerous mutually superposed components.

The document WO9806360 describes a nasal strip comprising three components, namely an elastically deformable element, a strip of soft flexible material, and a dermocompatible adhesive substance applied to a surface of said soft strip.

The elastically deformable element (with a dilator function) consists particularly of a bar which is joined to the soft flexible strip by welding, or by sandwiching it within a pocket provided in the soft flexible strip.

This known structure results in numerous drawbacks.

In this respect when in use, after being made to adhere to the surface of the nose of a user of the strip, the elastically deformable element has to outwardly raise (away from the nose surface) the soft flexible strip which, via the adhesive substance, dilates the nose nostrils.

Hence in practice, as this very complex structure damps the force transmitted by the elastically deformable element to the soft strip for dilating the nostrils (in this respect it has to outwardly raise the entire nose surface on which the soft material adheres), elastically deformable elements of high rigidity have to be used, with the result that the resistant force of the adhesive substance has to be increased, with consequent damage to the skin of the nose.

In this respect, as the adhesive substance is spread over the entire surface of the soft flexible strip, the traction exerted on this strip by the elastically deformable element is distributed over the entire strip surface in contact with the skin, so determining a reduction in the widening effect on the nostril by the nasal strip.

Finally, the use of three separate constituent components of the known nasal strip results in high costs.

### SUMMARY OF THE INVENTION

The technical aim of the present invention is therefore to provide a nasal dilator by which the stated technical drawbacks of the known art are eliminated.

Within the scope of this technical aim, an object of the invention is to provide a nasal dilator which can be applied directly (i.e. without interposing a strip of soft flexible material) to the surface of a user's nose to dilate the nostrils and facilitate respiration, without damaging the skin of the nose.

Another object of the invention is to provide a nasal dilator which can be applied to the skin very effectively, so enabling the force required to ensure dilation of the nostrils to be reduced (compared with that necessary with known nasal strips).

A further object of the invention is to provide a nasal dilator which is more economical than nasal strips of known type.

The technical aim, together with these and other objects, are attained according to the present invention by a nasal dilator consisting of an elongated elastically flexible element formed of plastic material and having a separate layer of dermocompatible adhesive material applied onto a first surface at each of those two ends of the element most distant apart. Preferably said element consists of a lamina of plastic material having a thickness less than 400 micron, advantageously between 80 and 300 micron; each of said layers of adhesive material is without solvents and has a thickness less than 120 micron; again preferably, said layers of adhesive material are permeable to vapour.

The aforesaid lamina of plastic material is preferably made of a material chosen from the group comprising polyester, PVC, polythene, polystyrene and nylon.

The dermocompatible adhesive material usable as a component of the nasal dilator is of known type, commonly used for adhesive plasters, i.e. consisting of a dermocompatible adhesive chosen from the group comprising water- or solvent-based acrylic adhesives, water or solvent based vinyl adhesives, polyurethane adhesives, resins of natural or synthetic origin, polyacrylates, natural polymers, gums, polyvinyl alcohols, cellulose, carrageens, and alginates.

Advantageously, as will be apparent from the examples of specific embodiments, the adhesive material, after its application to the support (i.e. on termination of the dilator preparation stage, comprising heating in an oven), is water or solvent-free and is transpirable and permeable to vapour and has (after evaporation of solvents in the oven) a thickness less than 120 micron and preferably between 20 and 80 micron.

The nasal dilator of the invention can also be usefully covered (on the surface opposite that to which the dermocompatible adhesive material is applied) by a layer of vapour-permeable acrylic resin containing an aromatic substance or essential oil, such that these substances are slowly released when the dilator is applied to the nose of a user.

### BRIEF DESCRIPTION OF THE DRAWING

The structure of the nasal dilator of the invention will be better understood from the description of a preferred embodiment thereof, with reference to the accompanying drawing, in which:

FIG. 1 is a plan view of a nasal dilator according to the invention;

FIG. 2 is a side view of the nasal dilator of FIG. 1, ready to be applied to a user's nose;

FIG. 3 shows the dilator of FIG. 2 provided with a removable layer of siliconized paper protecting the adhesive material applied to the ends of a surface of the nasal dilator; and

FIG. 4 is a side view of a second embodiment of a nasal dilator according to the invention provided, on a surface thereof opposite that comprising the adhesive material, with a layer of material containing an aromatic substance or essential oil;

### DETAILED DESCRIPTION OF THE INVENTION

The figures show a nasal dilator indicated overall by the reference numeral 1.

The nasal dilator 1 consists of an elastically yieldable elongated support lamina 2 having two separate adhesive layers on a surface 4 thereof.

Specifically, as shown in the Figures from 2 to 4, the nasal dilator presents two separate adhesive layers 7, 8 applied to the two opposing ends of the surface 4 of the lamina 2: these layers 7, 8 cover (when the dilator is in use) only a small portion of the nose surface, with consequent benefits in terms of tolerability and transpirability.

From FIG. 3 it can be seen that a sheet 11 (for example of siliconized paper, as known in the art) is applied to the adhesive material layers 7, 8 and has to be removed before using the nasal dilator 1.

FIG. 4 shows a more complex embodiment of the dilator, in which on that surface 12 of the lamina opposite the surface 4 on which the adhesive layers 7, 8 are applied, a layer 15 of a material (preferably an acrylic resin) containing an aromatic substance or essential oil is applied; this layer 15 is also free of solvents and is permeable to vapours (on termination of the application stage comprising heating in an oven, as described hereinafter).

For use, a layer 11 protecting the adhesive 7, 8 is removed prior to use of the dilator, and the strip 1 is applied straddling the user's nose, with the adhesive layers 7, 8 pressed down and fixed in positions correspond with the nasal nostrils: the small size of the layers 7 and 8 leaves most of the surface of the nasal skin completely free.

The support 2 flexes when applied to the nose and tends, by virtue of its elasticity, to return to a straight configuration, and dilate the nostrils. If present, the layer 15 releases the substances dispersed therein to gradually evolve aromatic and balsamic vapours, for example to facilitate respiration.

The following Examples describe the method of preparing nasal dilators with reference to the aforedescribed figures of the accompanying drawings.

### EXAMPLE 1

Preparation of a Nasal Dilator Comprising a Polyester Lamina of 100 Micron Thickness.

15.18 kg of solvent based acrylic adhesive (for example adhesive produced and sold under the name Durotak 2819 of National Starch & Chemical Co.) are fed cold into a container.

Using a patch spreading machine and with the aid of a compressed air pump the acrylic adhesive mixture is transferred onto the rotating cylinder doctor, having adjusted the doctor thickness to about 200 micron.

The doctor spreads the adhesive (to the set thickness) onto a continuous plastic web.

To form nasal dilators having only their ends provided with the adhesive layer, the adhesive is prevented from being spread over the entire surface of the plastic lamina by providing the doctor (in known manner) with dividers to form numerous doctor scrapers, each of which deposits on the plastic lamina a 40 mm band of adhesive, leaving 20 mm free of adhesive. Deposition continues for the entire length of the plastic web on which continuous adhesive bands of 40 mm width are hence present spaced apart by empty distances (i.e. without adhesive) of 20 mm.

The plastic web on which the adhesive is spread is formed of 100 micron thick polyester. After the adhesive has been spread in bands on this web, the web is passed at a speed of 8 metres/minute through four successive oven stations, the first oven station having its temperature controlled at 40° C., the second at 50° C., the third at 70° C. and the fourth at 80° C.

At the oven exit the adhesive layers are completely free of solvents, which have evaporated in the oven stations; the thickness of the adhesive mass is about 41 g/m².

At the oven exit a strip of 80 g/m² siliconized paper is applied and pressed onto the surface of the polyester web where the adhesive material bands are provided, hence forming a combination which is rewound into a reel. The result is that the adhesive layers, protected by the siliconized paper, bind to the polyester lamina.

The composite reel obtained is then cut into reels of lesser dimensions by cutting the 40 mm adhesive layers in half to obtain narrower reels of total width 60 mm, in which the side 20 mm portions are provided with an adhesive layer.

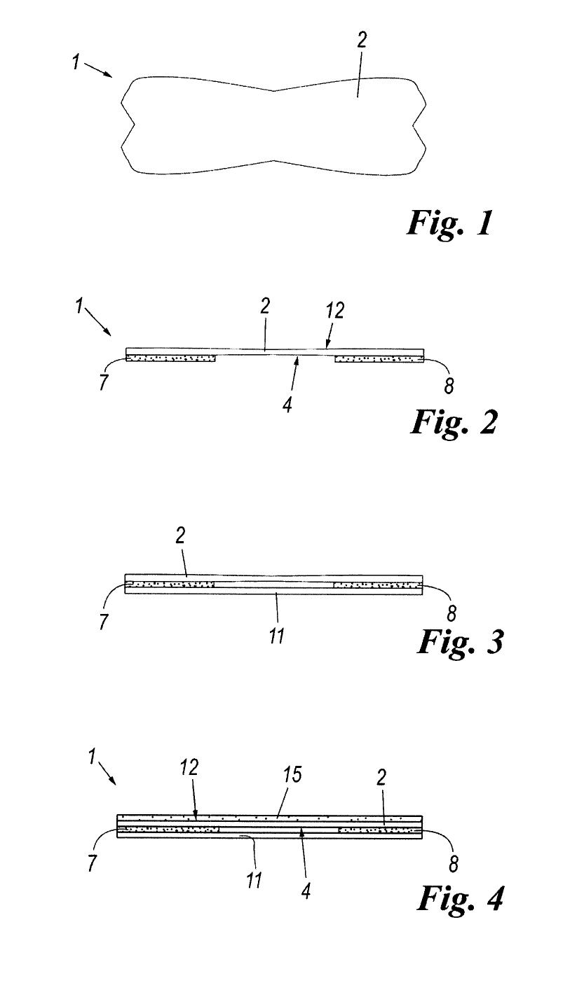

These reels are then cut and shaped by a punching machine as shown in FIG. 1, to give nasal dilators protected by siliconized paper, which are then packaged.

A nasal dilator is shown in plan view in FIG. 1 in which the numeral 1 indicates the dilator and the numeral 2 indicates the polyester lamina; in FIG. 2 which shows the nasal dilator in side view, in which two separate portions 7 and respectively 8 of adhesive can be seen applied to the ends of the lamina 2; and in FIG. 3 (similar to FIG. 2) but in which the adhesive material 7, 8 is protected by a profiled sheet 11 of siliconized paper.

### EXAMPLE 2

Preparation of a Nasal Dilator Formed with a Polyester Lamina of 150 Micron Thickness.

30 kg of water based acrylic adhesive (methyl acrylate polymer—for example adhesive known by the registered name ACRONAL-500 D adhesive) are fed cold into a container.

The preparation proceeds as indicated for example 1.

Using a patch spreading machine and with the aid of a compressed air pump the adhesive is transferred onto the rotating cylinder doctor, having adjusted the doctor thickness to about 200 micron; by means of the doctor the adhesive is applied to a web of plastic material.

To prevent the adhesive from being spread over the entire surface of the web, various doctor scrapers are formed, each of which deposits a band of 40 mm of adhesive, leaving 20 mm free of adhesive, and so on for the entire width of the polyester web having a thickness of 150 micron polyester.

The adhesive spread in bands on the polyester web passes through 4 oven stations, the first oven being controlled at 120° C., the second at 130° C., the third at 100° C. and the fourth at 90° C., with a speed of 8 metres per minute.

At the oven exit the adhesive layers are completely free of solvents, which have evaporated in the oven stations; the thickness of the adhesive mass is about 50 g/m².

The polyester web is coupled to siliconized paper of 80 g/m² and rewound into a reel. The result is that the adhesive layers, protected and pressed by the siliconized paper, bind to the polyester web.

The reel obtained is then cut into reels of lesser dimensions by cutting the 40 mm adhesive layers in half to obtain reels of total width 60 mm, in which the side 20 mm portions are provided with an adhesive layer.

The rolled-up webs obtained in this manner are then cut and shaped by a punching machine to give nasal dilators (as shown in Figures from 1 to 3), which are then packaged.

### EXAMPLE 3

Preparation of a Nasal Dilator Formed with a PVC Lamina of 200 micron thickness.

50 kg of solvent based acrylic adhesive (for example Duro-tak-280A adhesive of National Starch & Chemical Co.) are fed cold into a container.

The preparation proceeds as indicated in Example 1, but adjusting the doctor thickness to about 360 micron and spreading the adhesive on a PVC web of 200 micron thickness and then passing the adhesive spread on PVC through four oven stations, the first being controlled at 60° C., the second at 70° C., the third at 80° C. and the fourth at 90° C., with a speed of 8 metres per minute.

At the oven exit the adhesive is completely free of solvents, which have evaporated in the oven stations; the thickness of the adhesive mass is about 50 g/m².

The PVC is then coupled to siliconized paper of 80 g/m² and rewound into a reel. The result is that the adhesive layers, pressed and protected by the siliconized paper, bind to the PVC.

The reel obtained in this manner is then cut into reels of lesser width by cutting the 40 mm adhesive layers in half to obtain reels of total width 60 mm, in which the side 20 mm portions are provided with an adhesive layer.

Finally, the rolled-up adhesive strips are shaped by a punching machine as shown in Figures from 1 to 3, and are then packaged.

### EXAMPLE 4

Preparation of a Nasal Dilator of Balsamic Action Formed with a Polyester Lamina of 100 Micron Thickness.

15.18 kg of solvent based acrylic adhesive (for example Duro-tak-2819 adhesive of National Starch & Chemical Co.) are fed cold into a container.

The preparation proceeds as already indicated in example 1.

Using an adhesive plaster spreading machine and with the aid of a compressed air pump the mixture is fed onto the rotating cylinder doctor, having adjusted the doctor thickness to about 200 micron. In the same manner as described in Example 1, separate continuous bands of adhesive are deposited on a surface of a continuous polyester web of thickness 100 micron; this web is then passed through four oven stations, in the same manner and with the same characteristics as already described.

At the oven exit the polyester web is press-coupled to 80 g/m² siliconized paper and rewound into a reel. In this manner a composite web is obtained in which the adhesive bands, protected by the siliconized paper, grip the surface of the polyester web which is then rewound to form a reel. Finally, on the other surface of the polyester web (that on which the adhesive bands are not present, a mixture is spread prepared in the following manner.

20 kg of a binder consisting of an aqueous dispersion of an acrylic and vinyl ester-based polymer (for example Binder 9011 produced and marketed by the company ICMA) are fed into an agitator, heated to 30° C. and mixed very slowly. 1.5 kg of eucalyptol essential oils and 0.5 kg of mint essential oil are fed in. These are mixed together for 10 minutes and then left standing for 30 minutes.

Using a transfer pump, this mixture is transferred onto a doctor blade set at 300 micron and spread over said other surface of the polyester web on that surface of the polyester web free of adhesive and not covered by the siliconized paper.

The composite web obtained in this manner is then passed through three separate successive oven stations, the first oven being controlled at 120° C., the second at 110° C., the third at 100° C., with a speed of 6 metres per minute. At the oven exit this mixture layer containing essential oils is completely free of water, which has evaporated in the oven stations, while the essential oils remain trapped in the binder; the thickness of the mass is about 100 g/m².

At the oven exit the polyester web, coupled on one side to the siliconized paper and covered on the other side with the aromatic resin, is rewound into a reel.

This reel is then cut into reels of lesser dimensions by cutting the 40 mm adhesive layers in half to obtain reels of total width 60 mm, in which the side 20 mm portions are provided with an adhesive layer.

The composite web obtained in this manner is then unwound from the reel and made to pass through a punching machine, where profiled nasal dilators are cut out (as shown in Figure) and are then immediately packaged. Each nasal dilator obtained in Example 4 comprises a profiled polyester lamina 2, on one surface 4 of which (and at the two ends of the lamina) are applied two separate portions 7 and respectively 8 of adhesive protected by a shaped sheet 11 of siliconized paper, while on the other surface (indicated by the numeral 112) of the other surface of the polyester lamina 2 a layer 15 of the mixture containing essential oils is applied.

Each nasal dilator, when applied to a person's nose, simultaneously performs two activities: it dilates the nostrils while slowly releasing balsamic aromas useful for example in the case of a cold.

What is claimed is:

1. A nasal dilator consisting of an elongated elastically flexible element formed of plastic material and having a separate layer of dermocompatible adhesive material applied onto a first surface thereof at each of those two ends of the element most distant apart.

2. A nasal dilator as claimed in claim 1, wherein said element consists of a lamina of plastic material.

3. A nasal dilator as claimed in claim 2, wherein said lamina of plastic material has a thickness less than 400 micron.

4. A nasal dilator as claimed in claim 3, wherein said lamina of plastic material has a thickness between 80 and 300 micron.

5. A nasal dilator as claimed in claim 1, wherein each of said layers of adhesive material is without solvents and has a thickness less than 120 micron.

6. A nasal dilator as claimed in claim 5, wherein said layers of adhesive material are permeable to vapour.

7. A nasal dilator as claimed in claim 6, wherein a layer of an acrylic and/or vinyl resin in which at least one aromatic substance is dispersed is applied to that surface of said lamina opposite said first surface to which a layer of adhesive material is applied.

8. A nasal dilator as claimed in claim 7, wherein said layer of acrylic resin in which said at least one aromatic substance is dispersed is without solvents and is permeable to vapour.
